(19) United States
(12) Patent Application Publication
(10) Pub. No.: US 2026/0053589 A1
(43) Pub. Date: Feb. 26, 2026

(54) TRACTION DRIVE AND INSERTION MONITORING FOR A FLEXIBLE DEVICE

(71) Applicant: Intuitive Surgical Operations, Inc., Sunnyvale, CA (US)

(72) Inventors: Matthew R. Cavalier, Los Gatos, CA (US); Alexander K. Sang, Blacksburg, VA (US); David W. Bailey, Portola Valley, CA (US)

(73) Assignee: Intuitive Surgical Operations, Inc., Sunnyvale, CA (US)

(21) Appl. No.: 19/310,604

(22) Filed: Aug. 26, 2025

Related U.S. Application Data
(60) Provisional application No. 63/686,985, filed on Aug. 26, 2024.

(51) Int. Cl. A61B 34/00; A61B 34/20; A61B 34/30; A61M 25/01

(52) U.S. Cl. CPC A61B 34/71; A61B 2034/2061; A61B 2034/301; A61M 25/0113

(57) ABSTRACT

A medical system includes a manipulator assembly that includes a traction drive for driving a flexible elongate device of a medical instrument along an insertion axis of the manipulator assembly. The traction drive is configured to form a detection zone that imprints a mechanical signature on the flexible elongate device. The manipulator assembly further includes a shape sensor configured to measure a shape of the flexible elongate device and a control system coupled to the manipulator assembly. The control system is configured to identify, using the shape sensor, the mechanical signature at a location of the flexible elongate device, and determine, based on the location, a spatial relationship between the flexible elongate device and the traction drive.

### CROSS-REFERENCE TO RELATED APPLICATIONS

This application claims priority to and the benefit of the filing date of U.S. Provisional Patent Application 63/686,985, filed on Aug. 26, 2024, which is hereby incorporated by reference herein in its entirety.

### FIELD

Disclosed embodiments relate to improved robotic and/or medical devices, systems, and methods.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter or bronchoscope, that can be inserted into anatomic passageways and navigated toward a region of interest within the patient anatomy.

Different anatomic passageways are of different lengths. For example, a human lung is approximately 25 cm long (from apex to base) while the small intestine is approximately 3 to 5 m. Therefore, a flexible elongate device suitable for the esophagus may not be suitable in terms of length for use in the bowel. Under some operating conditions, a medical instrument may be driven along one or more movement axes, to advance the elongate device into anatomic passageways. A robotic drive for inserting a flexible elongate device into anatomic passageways must be capable of facilitating the insertion of different length devices into anatomic passageways of different lengths/depths. To facilitate a medical technique (for example surgical, diagnostic, therapeutic, and/or biopsy), it is necessary to control the insertion of the flexible elongate device so that the medical technique may be performed on the correct location of the anatomic passageway.

### SUMMARY

The following presents a simplified summary of various examples described herein and is not intended to identify key or critical elements or to delineate the scope of the claims.

In some examples, a medical system comprises: a manipulator assembly comprising: a traction drive for driving a flexible elongate device of a medical instrument along an insertion axis of the manipulator assembly, wherein the traction drive is configured to form a detection zone that imprints a mechanical signature on the flexible elongate device; a shape sensor configured to measure a shape of the flexible elongate device; and a control system coupled to the manipulator assembly, the control system configured to: identify, using the shape sensor, the mechanical signature at a location of the flexible elongate device, and determine, based on the location, a spatial relationship between the flexible elongate device and the traction drive.

In some examples, a non-transitory machine-readable medium comprises a plurality of machine-readable instructions executed by one or more processors associated with a medical system, the plurality of machine-readable instructions causing the one or more processors to perform a method comprising: identifying, using a shape sensor configured to measure a shape of a flexible elongate device, a mechanical signature at a location of the flexible elongate device, the mechanical signature being imprinted on the flexible elongate device by a detection zone of a traction drive comprised in a manipulator assembly, wherein the traction drive is configured to driving the flexible elongate device of a medical instrument along an insertion axis of the manipulator assembly; and based on the location, determining a spatial relationship between the flexible elongate device and the traction drive.

In some examples, a method of operating a medical system comprises: identifying, using a shape sensor configured to measure a shape of a flexible elongate device, a mechanical signature at a location of the flexible elongate device, the mechanical signature being imprinted on the flexible elongate device by a detection zone of a traction drive comprised in a manipulator assembly, wherein the traction drive is configured to driving the flexible elongate device of a medical instrument along an insertion axis of the manipulator assembly; and based on the location, determining a spatial relationship between the flexible elongate device and the traction drive.

In some examples, a traction drive for driving a flexible elongate device comprises: a first clamping stage configured to releasably clamp the flexible elongate device, the first clamping stage comprising: an orifice for the flexible elongate device, wherein the orifice has an adjustable aperture and an adjustable orientation; and a first driving stage configured to move the first clamping stage along a longitudinal axis of the flexible elongate device.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within a flexible elongate device according to some embodiments.

FIG. 3 is a simplified diagram of a side view of a patient coordinate space including a medical system according to some embodiments.

FIG. 4 is an illustration of a manipulator assembly according to some embodiments.

FIG. 5A is an illustration of an example roller configuration in a traction drive, according to some embodiments.

FIG. 5B is an illustration of a plot of the shape of the flexible elongate device against length along the flexible elongate device using the roller configurations of FIG. 5A, according to some embodiments.

FIG. 6A is an illustration of an example roller configuration in a traction drive, according to some embodiments.

FIG. 6B is an illustration of a plot of the shape of the flexible elongate device against length along the flexible elongate device using the roller configurations of FIG. 6A, according to some embodiments.

FIG. 6C is an illustration of a plot of the strain of the flexible elongate device against length along the flexible elongate device using the roller configurations of FIG. 6A, according to some embodiments.

FIG. 7 is a flowchart of a method according to some embodiments.

FIGS. 8A and 8B are illustrations of traction drives according to some embodiments.

FIGS. 9A and 9B are illustrations of clamping stages according to some embodiments.

FIG. 10 is an illustration of a traction dive according to some embodiments.

FIG. 11 is an illustration of a traction drive according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Embodiments of the disclosure include medical systems and methods for operating such medical systems. Medical systems may be medical systems that use flexible elongate devices (e.g., catheters, bronchoscopes, endoscopes, etc.), but also other medical systems.

A medical system may couple to a medical instrument to cause the medical instrument to be driven along one or more movement axes, while determining a spatial relationship between the medical instrument and a component of the medical system.

A more detailed discussion of the medical system is provided below in reference to the figures.

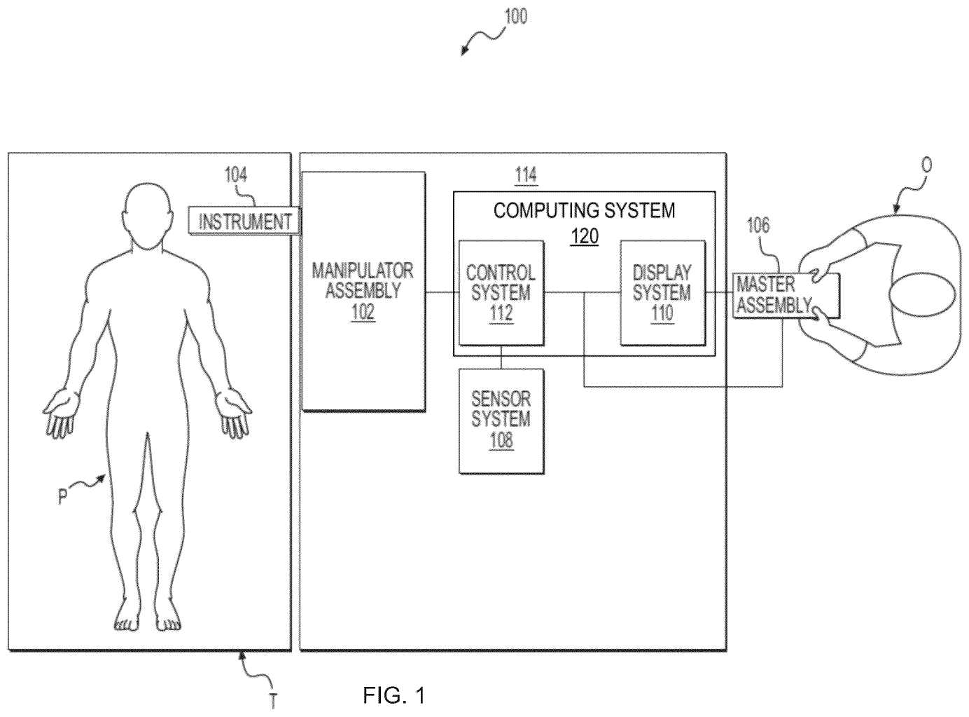

Turning to the figures, FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be manually controlled by the operator O. Direct operator control may include various handles and operator interfaces for hand-held operation of the medical instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo-controlled links (e.g., one or more links that may be manually positioned and locked in place) and/or one or more servo-controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016, and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011, and titled “Medical System Providing Dynamic of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

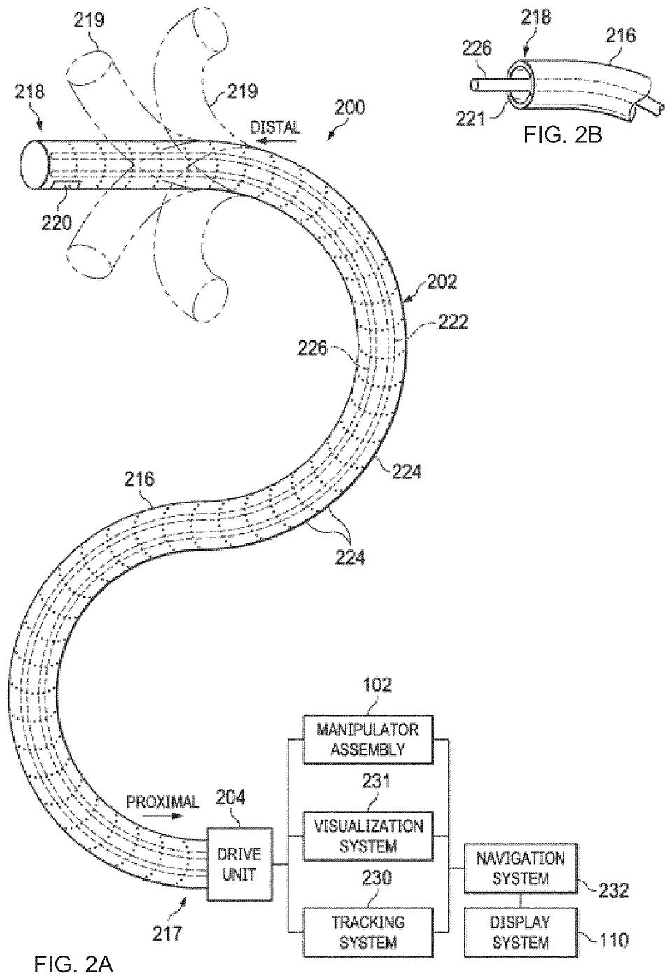

FIG. 2A is a simplified diagram of a medical instrument system 200 according to some embodiments. The medical instrument system 200 includes a flexible elongate device 202 (also referred to as elongate device 202), a drive unit 204, and a flexible tool, e.g., a medical tool, 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel or lumen 221 through which a flexible tool, e.g., the medical tool, 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexible body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999, and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of poses, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor system 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the flexible tool 226 within the elongate device 202 according to some embodiments. The flexible body 216 of the elongate device 202 may include the lumen 221 sized and shaped to receive the flexible tool 226. In some embodiments, the flexible tool 226 may be used for procedures such as diagnostics, imaging, surgery, biopsy, ablation, illumination, irrigation, suction, electroporation, etc. Flexible tool 226 can be deployed through channel or lumen 221 of flexible body 216 and operated at a procedural site within the anatomy. Flexible tool 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the flexible tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The flexible tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the lumen 221 when the biopsy tool is within the lumen 221. The flexible tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of flexible tool 226 to facilitate simultaneous image capture and tissue intervention, such as within the same lumen 221 or in separate channels. A flexible tool 226 may be advanced from the opening of the lumen 221 to perform the procedure (or some other functionality) and then retracted back into the lumen 221 when the procedure is complete. The flexible tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 215 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. Steerable instruments or tools, applicable in some embodiments, are further described in detail in U.S. Pat. No. 7,316,681 (filed on Oct. 4, 2005, and titled “Articulated Surgical Instrument for Performing Minimally Invasive Surgery with Enhanced Dexterity and Sensitivity”) and U.S. Pat. No. 9,259,274 (filed Sep. 30, 2008, and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019, and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. The drive unit 204 may further include brakes. One brake may be paired with one actuator. In configurations that pair an actuator with a gear reducer, the brake may be located on the actuator side, which enables even a relatively small brake to produce a significant braking force. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011, and titled “Medical System Providing Dynamic Registration of a Model of an Anatomic Structure for Image-Guided Surgery”), which is incorporated by reference herein in its entirety.

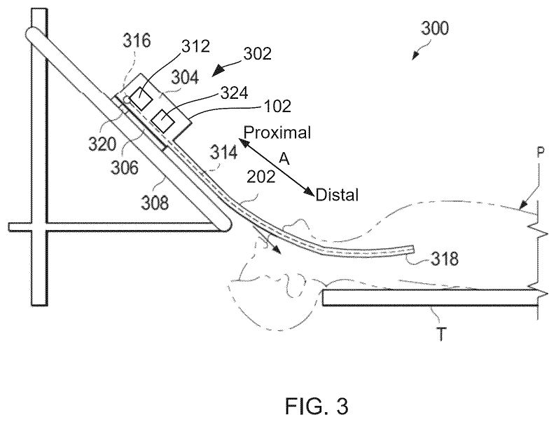

FIG. 3 is a simplified diagram of a side view of a patient coordinate space including elements of a medical system that includes the manipulator assembly 102. As shown in FIG. 3, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, the medical system comprising a medical instrument 304 and a flexible elongate device 202 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104.

Elongate device 202 may also include one or more sensors (e.g., components of the sensor system 108). In some examples, a shape sensor 314 may be fixed at a proximal point 316 on the instrument body 312. The shape sensor 314 may measure a shape from the proximal point 316 to another point, such as a distal end 318 of the elongate device 202. The shape sensor 314 may be aligned with the elongate device 202 (e.g., provided within an interior channel or mounted externally). In some examples, the shape sensor 314 may include optical fibers, used to generate shape information for the elongate device 202.

In some examples, position sensors (e.g., EM sensors) may be incorporated into the medical instrument 304. A series of position sensors may be positioned along the flexible elongate device 202 and used for shape sensing. Position sensors may be used alternatively to the shape sensor 314 or with the shape sensor 314, such as to improve the accuracy of shape sensing or to verify shape information.

Elongate device 202 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements e.g., integrated in drive unit 204, such as actuators, of a manipulator assembly.

In some embodiments of FIG. 3, the medical system 302 includes a traction drive 324 configured to drive the flexible elongate device 202 along axis A to provide for insertion and retraction of the flexible elongate device 202 with respect to the patient P anatomy. Additionally or alternatively, the traction drive 324 may be configured to drive the flexible elongate device 202 to roll about axis A for rotation of the flexible elongate device 202 with respect to the patient P anatomy.

In FIG. 3, the medical system 302 is shown as being mounted on a support structure 308. The support structure 308 may take other configurations than that illustrated in FIG. 3 and may be any type of mechanical support, including a robotic arm, a stationary or movable platform, etc.

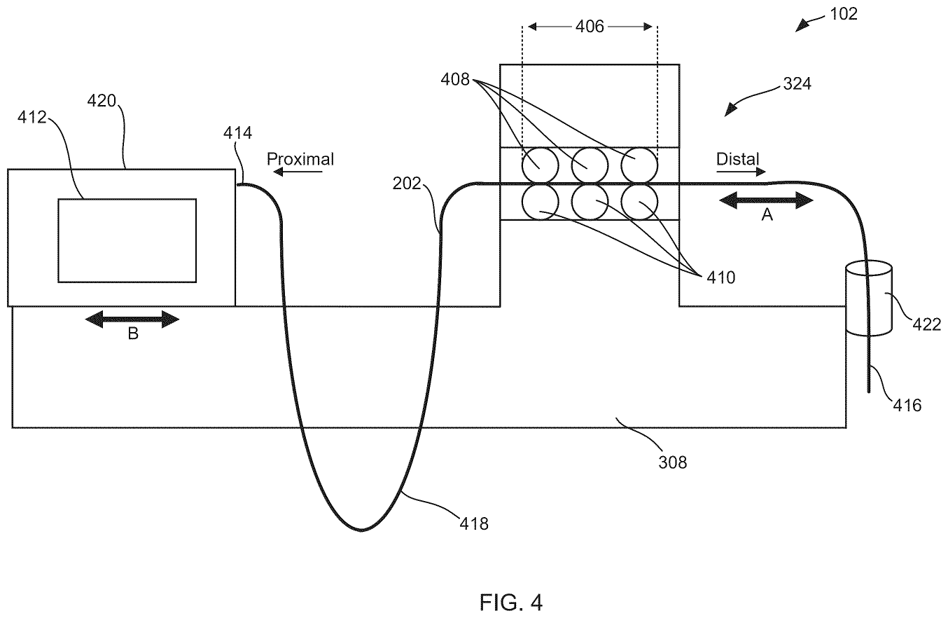

FIG. 4 illustrates a manipulator assembly 102 according to the present disclosure. The manipulator assembly 102 may be the manipulator assembly 102 of FIG. 1 or the manipulator assembly 102 of FIG. 3. The manipulator assembly 102 comprises a traction drive 324 (see FIG. 3) disposed on a support structure 308. The traction drive 324 is configured to move a flexible elongate device 202 so as to enable insertion and retraction of the flexible elongate device 202 with respect to the patient anatomy. The traction drive 324 is configured to mechanically couple to the flexible elongate device 202 so as to drive the flexible elongate device 202 along (and/or roll about) an insertion axis A of the manipulator assembly 102 using traction force. Traction can be defined as a physical process in which a tangential force is transmitted across an interface between two bodies resulting in motion, stoppage or the transmission of power. The transmission may occur through dry friction, an intervening fluid film, magnetic coupling, or other mechanisms.

The traction drive 324 is configured to form a detection zone 406 that imprints a mechanical signature on the flexible elongate device.

In some embodiments, the traction drive 324 comprises a first set of rollers 408 held in a fixed position on a first side of a portion of the flexible elongate device 202 that passes through the traction drive 324, and a second set of rollers 410 held in a fixed position on a second side of the portion of the flexible elongate device 202 that passes through the traction drive 324. The first set 408 and second set of rollers 410 are configured to mechanically couple to a portion of the flexible elongate device 202. While sets of three rollers are shown, the first and second sets of rollers 408 410 may include any number or rollers, without departing from the disclosure. The first and second sets of rollers may have the same or different number or rollers. Other roller 408 410 configurations are possible, and will be discussed later. In some embodiments, the first set 408 and second set of rollers 410 are configured to contact the portion of the flexible elongate device 202 that passes through the traction drive 324.

The first set 408 and second set 410 of rollers are configured to actively rotate, driven by one or more actuators (not shown) such as servomotors which may or may not include brakes, so that their movement causes a corresponding movement of the flexible elongate device 202 in a direction along the axis A. Power from the moving rollers 408 410 is transmitted from the moving rollers 408 410 to the passive flexible elongate device 202 causing it to move in a direction along the A axis.

Each roller in the first set 408 and second set 410 of rollers may be comprised of the same material, including metals, plastics and other composites. Alternatively, the rollers in the first set 408 and/or second set 410 may be comprised of different materials. In some embodiments, each roller in the first set 408 and second set 410 of rollers has a substantially smooth outer surface. In an alternative embodiment, each roller in the first set 408 and second set 410 of rollers has a substantially textured outer surface configured to couple to the flexible elongate device 202.

In the embodiment of FIG. 4, the detection zone 406 is defined as a region where the rollers 408 410 are configured to couple to the flexible elongate device 202 so as to imprint the mechanical signature. Different types of mechanical signatures may be used. The mechanical signatures will be discussed further in the description.

The flexible elongate device 202 is equipped with a shape sensor (not shown). The shape sensor may be the shape sensor 222 of FIG. 2A or the shape sensor 314 of FIG. 3, and comprise any of the techniques detailed previously. The shape sensor is configured to electronically couple to a control system (not shown), e.g., control system 112 of FIG. 1. The control system may comprise the sensor system 108 of FIG. 1, including a shape measurement system. The control system is configured to identify the mechanical signature at a location on the flexible elongate device 202 using the shape sensor. The location on the flexible elongate device may be in relation to a distal end 416 or a proximal end 414 of the flexible elongate device 202 which terminates at the instrument body 412.

Based on determining the location of the mechanical signature along the flexible elongate device 202, a spatial relationship between the flexible elongate device 202 and the traction drive 324 may be identified. In some embodiments, the length of a segment of the flexible elongate device 202 distal to the detection zone 406 may be determined. In a retracted position, the distal end 416 of the flexible elongate device 202 may be positioned just inside an entry orifice of a patient. In this retracted position, the location of the mechanical signature along the flexible elongate device may be at a particular location, which may correspond to a zero value of and/or other reference value of an insertion depth. Further insertion of the flexible elongate device 202, i.e., an increase in the insertion depth, reduces the length of the segment proximal to the detection zone and increases the length of the segment of the flexible elongate device distal to the detection zone increases by the same amount. Further, a registration procedure may be performed, as detailed previously, to determine the location of the flexible elongate device 202 relative to the patient anatomy, e.g., based on a model of the patient anatomy and the known length of the segment of the flexible elongate device distal to the detection zone. The model of the patient anatomy may have been generated from CT images or other data.

Using the approach as described, the insertion depth may, thus, be determined for a flexible elongate device 202 of any length. This approach as described overcomes the problem of slip associated with the use of traction drives for accurately controlling insertion and retraction of flexible elongate devices. While slip may occur in the traction drive, this does not negatively affect the accuracy of the insertion, as a spatial relationship between the traction drive and the flexible elongate device is monitored based on the location of the mechanical signature along the flexible elongate device.

In some embodiments, the flexible elongate device 202 is in a slack configuration. In some embodiments, the flexible elongate device 202 comprises a slack loop 418, where the flexible elongate device 202 is allowed to hang in a slack (loose or unsupported) manner.

The slack loop 418 accommodates the use of flexible elongate devices 202 that extend beyond the standard length. In FIG. 4, the slack loop 418 is located between a proximal end 414 of the flexible elongate device 202 at the instrument body 412 and the traction drive 324. This configuration results in movement of the traction drive 324 causing movement of the distal end 416 of flexible elongate device 202 (e.g., inside the patient) along axis A, even in the presence of slack. Additionally, any movement (intentional or non-intentional) of the instrument body 412 will not be translated into movement of the distal end 416 of the flexible elongate device 202.

By using a slack configuration as a feature to accommodate extra length, flexible elongate devices of any length may be used without requiring a corresponding increase in size of the medical system. This enables use of flexible elongate devices in very different environments such as, for example, a human lung that may be assumed to be approximately 25 cm long (from apex to base) vs the small intestine that may be assumed to be approximately 3 to 5 m long.

In some embodiments, the traction drive 324 pushes the flexible elongate device 202 through a swivel connector 422 to the patient. In some embodiments, the swivel connector is comprised in the manipulator assembly 102. The swivel connector 422 is located distal to the traction drive 324 close (or proximal) to the entry orifice of a patient P. The swivel connector 422 receives the flexible elongate device 202 and provides mechanical guidance at the interface to a working lumen near at an orifice. For example, in case of the working lumen being the airways of a lung, the orifice may be the patient's mouth, and the swivel connector may be positioned near the mouth to provide mechanical guidance of the flexible elongate device during insertion and retraction of the flexible elongate device. The swivel connector 422 prevents twisting, tangling and/or kinking/bending of the flexible elongate device 202. The swivel connector 422 may further be used to imprint a mechanical signature on the flexible elongate device 202, either in addition to or as an alternative to the signature imprinted by the rollers 408, 410 of the traction drive 324. Such a configuration may help improve accuracy. The swivel connector 422 may have a characteristic shape to imprint the mechanical signature. For example, the swivel connector 422 may include a straight section, a curved section, etc. that is detectable using the shape sensor 314.

In some embodiments, the instrument body 412 is stationary relative to the support structure 308. In some embodiments, the manipulator assembly 102 includes an instrument carriage 420 movably disposed on the support structure 308, e.g., using linear tracks. The instrument carriage may be configured to move the instrument body 412 along the support structure 308 along the B axis. The instrument carriage 420 may be used to maximize the length of the flexible elongate device 202 available for insertion. Specifically, when, during the insertion, the slack from the slack loop 418 has mostly or completely been consumed as a result of the insertion, the instrument carriage 420 may move along the B axis towards the traction drive 324, thereby making an additional section of the flexible elongate device 202 available for insertion. More generally, the instrument carriage 420 may enable a mechanical reconfiguration through translation, pivoting, etc., to provide an additional segment of flexible elongate device available for insertion. The mechanical reconfiguration may be performed either actively by an actuator or passively by a user of the medical system. In the embodiment where the manipulator assembly 102 includes an instrument carriage 420, the proximal point 316 of the shape sensor 314 may be movable with the instrument body 312, and the location of the proximal point 316 with respect to a desired reference frame may be known (e.g., via a tracking sensor or other tracking device).

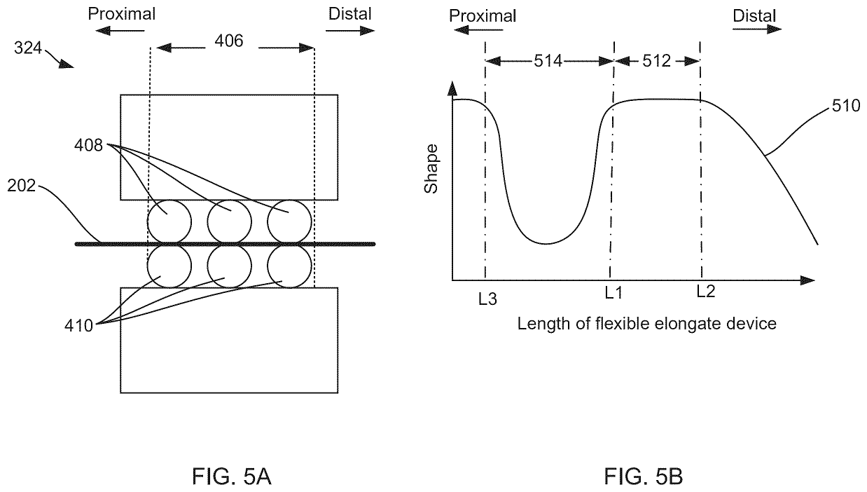

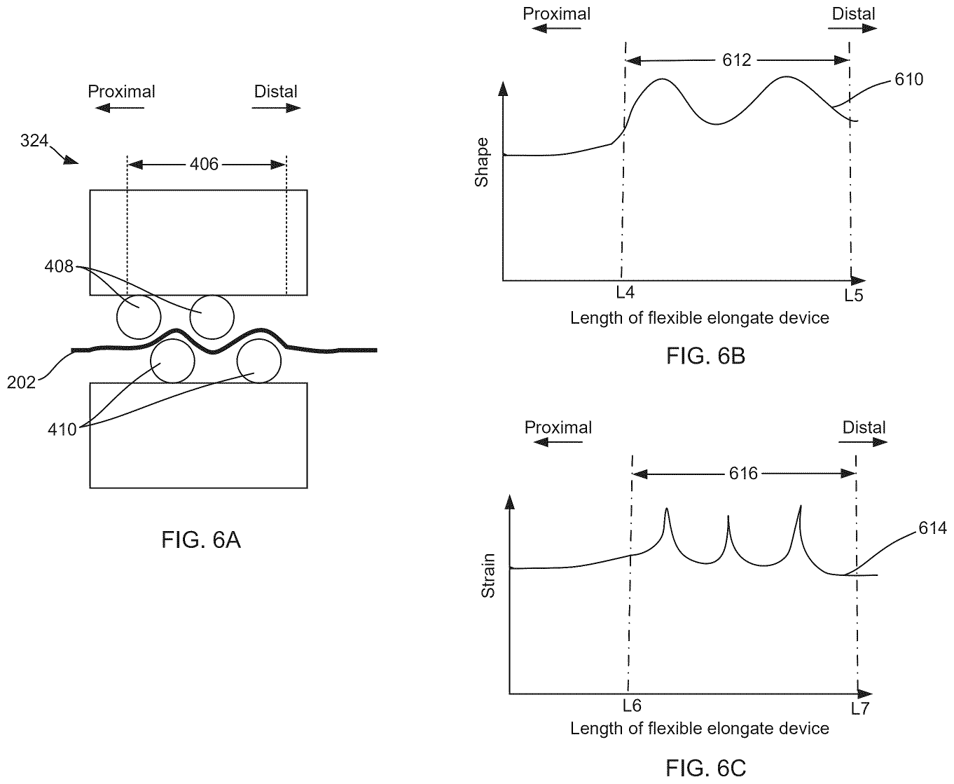

FIG. 5A and FIG. 6A illustrate example roller configurations in a traction drive 324, according to embodiments, where the traction drive is the traction drive 324 of FIG. 4. FIG. 5B and FIG. 6B illustrate plots of the shape of the flexible elongate device against length along the flexible elongate device using the roller configurations of FIG. 5A and FIG. 6A respectively.

FIG. 5A illustrates the traction drive 324 comprising a first set of rollers 408 and a second set of rollers 410 arranged to form a detection zone 406 with a straight section, i.e. the portion of the flexible elongate device 202 is kept substantially straight within the detection zone 406. This configuration has previously been described with reference to FIG. 4. In the embodiment of FIG. 5A, it is the straight section within the detection zone 406 that is the mechanical signature. The straight section can then be detected by the control system (112 in FIG. 1).

FIG. 5B illustrates a plot 510 of the shape of the flexible elongate device 202 against length along the flexible elongate device 202 using the roller configuration of FIG. 5A. The shape of the flexible elongate device 202 is measured by the control system 112 in conjunction with the shape sensor. The shape of the flexible elongate device 202 changes along the length of the flexible elongate device 202. In the portion 512 of the plot 510, the plot 510 of shape against length is substantially straight for a distance that substantially corresponds to the length of the detection zone 406. Therefore, from the control system 112 and the shape senor, it can be identified that the portion of the flexible elongate device 202 corresponding to distance L1 to L2 also is within the detection zone 406 of the traction drive 324. In this way, a spatial relationship between the flexible elongate device 202 and the traction drive 324 may be established. In some embodiments, the output of the shape sensor measured by the control system 112 may be differentiated to determine a zero-change of the shape over the straight detection zone 406.

In the plot 510 of FIG. 5B, it is also possible to identify a slack loop region of the flexible elongate device when a slack loop (418 of FIG. 4) is present.

As illustrated in FIG. 6A, in an alternative embodiment, the rollers may imprint a pattern on the flexible elongate device 202. FIG. 6A illustrates the traction drive 324 comprising a first set of rollers 408 and a second set of rollers 410 arranged to form a detection zone 406. As illustrated in FIG. 6A, the first set of rollers 408 and the second set 410 of rollers are arranged to force the flexible elongate device 202 to follow a substantially zig-zag or sinusoidal pattern in the detection zone 406. In the embodiment of FIG. 6A, it is the zig-zag or sinusoidal section within the detection zone 406 that is the mechanical signature. The zig-zag or sinusoidal section can then be detected by the control system 112 in conjunction with the shape sensor. Depending on the configuration of the rollers 408, 410. The traction drive 324 causes a detectable shape change or a detectable strain in the flexible elongate device 202. Smaller rollers that cause sharper directional changes (e.g., a shown in FIG. 6A) are more likely to cause a strain, whereas larger rollers that cause more gradual directional changes are more likely to cause a shape change. Additional details are provided below in reference to FIGS. 6B and 6C.

FIG. 6B illustrates a plot 610 of the shape of the flexible elongate device 202 against length along the flexible elongate device 202 using the roller configuration of FIG. 6A. The shape of the flexible elongate device 202 is measured by the control system 112 in conjunction with the shape sensor. The shape of the flexible elongate device 202 changes along the length of the flexible elongate device 202. In the portion 612 of the plot 610, the plot 610 of shape against length is substantially zig-zag or sinusoidal for a distance that substantially corresponds to the length of the detection zone 406. Therefore, from the control system 112 and the shape sensor, it can be identified that the portion of the flexible elongate device 202 corresponding to distance L4 to L5 is within the detection zone 406 of the traction drive 324. In this way, a spatial relationship between the flexible elongate device 202 and the traction drive 324. In some embodiments, the output of the shape sensor measured by the control system 112 may be differentiated to a rate of change of the shape to identify the zig-zag or sinusoidal mechanical signature.

FIG. 6C illustrates a plot 614 of strain of the flexible elongate device 202 against length along the flexible elongate device 202 using the roller configuration of FIG. 6A. The strain may be obtained using the shape sensor in conjunction with the control system 112. The strain plot 614 demonstrates a distinct spikey signature in the detection region 616, caused by contact of the rollers 408 410 with the flexible elongate device 202. Spikey signatures may be a result of increased deformation of the shape sensor caused by, for example, roller configurations that cause sharper directional changes, etc.

While the pattern of the mechanical signature imprinted on the flexible elongate device 202 in FIG. 6A is described as being zig-zag or sinusoidal, it will be understood that other patterns are achievable through different configuration of the rollers 408 410.

In another embodiment, some of the rollers of the first set of rollers 408 or the second set of rollers 410 may be textured. The texture may take the form of gear teeth, ridges or another form. The textured rollers may impart a distinguishable mechanical signature on the flexible elongate device 202. Specifically, the texture may impose particularly sharp small-scale shape changes that, as previously described, result in detectable strain.

Referring to FIG. 4, in some embodiments, by identifying a length of the flexible elongate device 202 within the detection zone 406, the control system 112 can determine a length of the flexible elongate device 202 distal to the traction drive 324 and/or a length of the flexible elongate device 202 proximal to the traction drive 324. In some embodiments, using a known distance from the traction drive 324 to the patient P entry orifice, a length of the flexible elongate device 202 within the patient P can be determined. In some embodiments, using knowledge about the anatomy of the anatomical passageway, the exact location of the flexible elongate device within the body may be determined. Further, a registration procedure, as described previously, may be performed to determine the location of the flexible elongate device relative to the patient anatomy, e.g., based on a model of the patient anatomy and the known length of the segment of the flexible elongate device distal to the detection zone. The model of the patient anatomy may have been generated from CT images or other data.

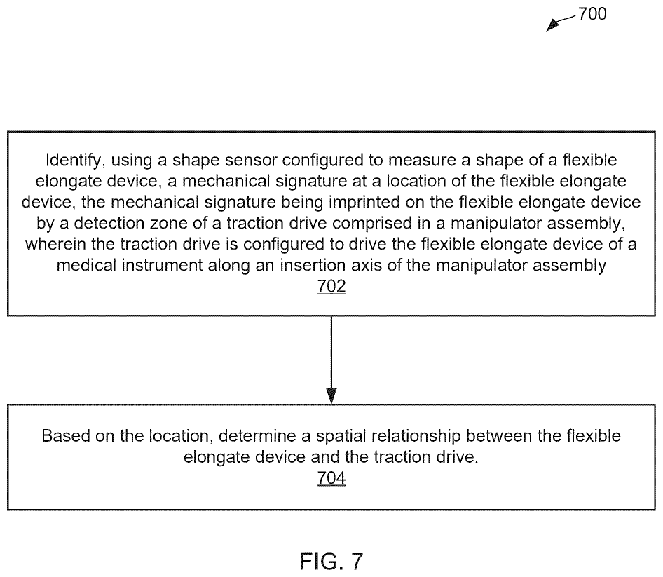

FIG. 7 shows a flowchart of a method 700 for operating a medical system, in accordance with embodiments of the disclosure. On a high level, the method 700 may be used to enable controlled insertion and retraction of a flexible elongate body.

The method may be implemented using instructions stored on a computer readable medium, for example, a non-transitory medium, that may be executed by a computing system, e.g., the computing system 120.

While the various blocks in FIG. 7 are presented and described sequentially, some or all of the blocks may be executed in different orders, may be combined or omitted, and some or all of the blocks may be executed in parallel. Furthermore, the blocks may be performed actively or passively.

In block 702, a mechanical signature at a location of a flexible elongate device is identified using a shape sensor configured to measure a shape of the flexible elongate device. The mechanical signature has been imprinted on the flexible elongate device by a detection zone of a traction drive comprised in a manipulator assembly. The traction drive is configured to drive the flexible elongate device of a medical instrument along an insertion axis of the manipulator assembly. The medical instrument, the manipulator assembly, the flexible elongate device, the shape sensor, the traction drive, the detection zone, and the mechanical signature have all been discussed previously with respect to any of the previous embodiments.

In block 704, based on the location determined in block 702, a spatial relationship between the flexible elongate device and the traction drive is determined. By identifying a length of the flexible elongate device 202 within the detection zone 406, the control system 112 can determine a length of the flexible elongate device 202 distal to the traction drive 324 and/or a length of the flexible elongate device 202 proximal to the traction drive 324. In some embodiments, using a known distance from the traction drive 324 to the patient P entry orifice, a length of the flexible elongate device 202 within the patient P can be determined. In some embodiments, using knowledge about the anatomy of the anatomical passageway, the exact location of the flexible elongate device within the body may be determined. Further, a registration procedure, as described previously, may be performed to determine the location of the flexible elongate device relative to the patient anatomy, e.g., based on a model of the patient anatomy and the known length of the segment of the flexible elongate device distal to the detection zone. The model of the patient anatomy may have been generated from CT images or other data.

Embodiments as described may use traction drives as subsequently discussed.

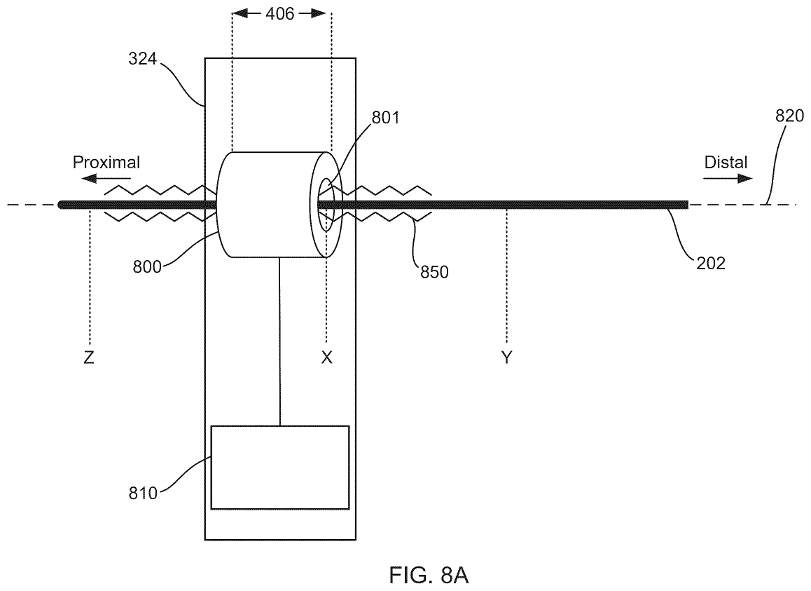

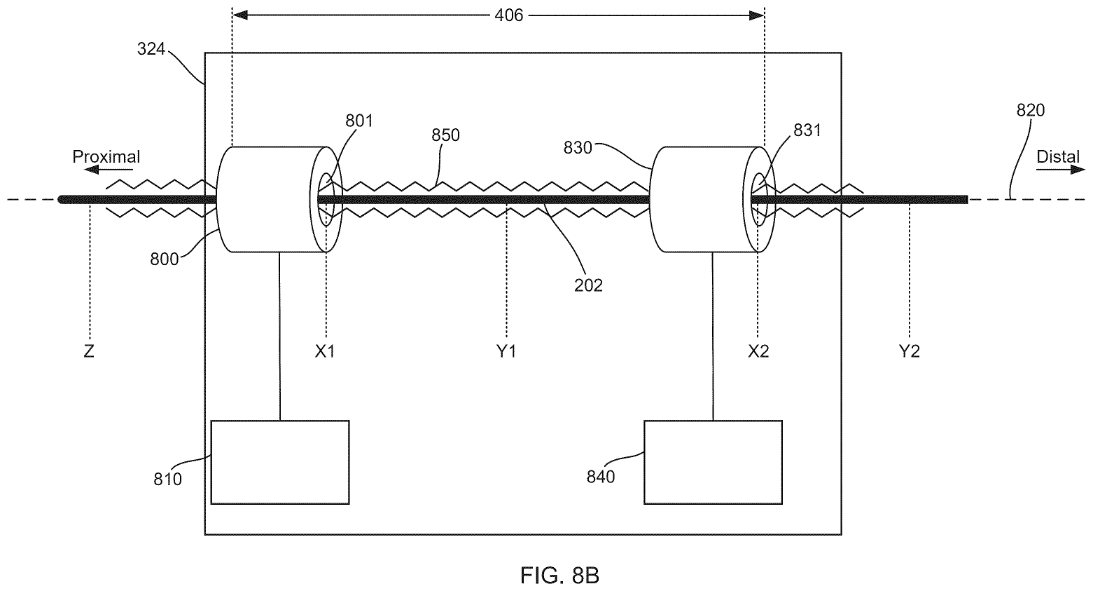

FIG. 8A illustrates an example configuration of a traction drive 324 for driving a flexible elongate device 202 according to some embodiments. FIG. 8A shows the traction drive 324 configured to couple to the flexible elongate device 202 to cause the flexible elongate device 202 to move in a direction along a longitudinal axis 820 associated with the flexible elongate device 202. In some embodiments, the longitudinal axis 820 may be substantially the same as the insertion axis A.

The traction drive 324 comprises a first clamping stage 800 and a first driving stage 810. The first clamping stage 800 is configured to releasably clamp the flexible elongate device 202. The first clamping stage 800 comprises an orifice 801 for the flexible elongate device 202. As illustrated in FIG. 8, the flexible elongate device 202 may pass through the orifice 801 of the first clamping stage 800. The orifice 801 is configured so that an aperture of the orifice 801 is adjustable so as to enable the first clamping stage 800 to clamp the flexible elongate device 202. The flexible elongate device 202 may be inserted into the orifice 801 of the first clamping stage 800 when the aperture has a diameter, or dimension, greater than a diameter, or dimension, of the flexible elongate device 202. Following insertion, the aperture of the orifice 801 may be adjusted so that the diameter, or dimension, of the aperture is such that the first clamping stage 800 clamps the flexible elongate device 202. The first clamping stage 800 may then be considered to be in a clamped state. While the orifice 801 is illustrated as being substantially cylindrical, it will be clear to the skilled person that other shaped orifices 801 could be used.

When the first clamping stage 800 is in a clamped state (clamping the flexible elongate device 202), a mechanical signature may be imprinted on the flexible elongate device 202 by the first clamping stage 800 and a detection zone 406 may be defined.

The first driving stage 810 is configured to move the first clamping stage 800 along a longitudinal axis 820 of the flexible elongate device 202. For example, when the flexible elongate device 202 is clamped by the first clamping stage 800, the first driving stage 810 causes the flexible elongate device 202 to move along the longitudinal axis 820 in a direction towards the procedural site (distal). Referring to FIG. 8A, the first driving stage 810 may cause the first clamping stage 800 to move from a position X to a position Y. The first clamping stage 800 may then release the flexible elongate device 202 by adjusting the aperture of the orifice 801 so that its diameter, or dimension, is greater than the diameter, or dimension, of the flexible elongate device 202. The first clamping stage 800 is then in an unclamped state (not clamping the flexible elongate device 202). In this way, a net movement of the flexible elongate device 202 by a distance illustrated between X and Y can be achieved. As a result, the traction drive 324 has generated an insertion movement for the flexible elongate device 202.

In a similar manner, the first driving stage 810 may cause the first clamping stage 800 to move along the longitudinal axis in a direction away from the procedural site (proximal). Referring to FIG. 8A, the first driving stage 810 may cause the first clamping stage 800, when in a clamped state, to move from a position X to a position Z. The first clamping stage 800 may then release the flexible elongate device 202 by adjusting the aperture of the orifice 801 so that its diameter, or dimension, is greater than the diameter, or dimension, of the flexible elongate device 202. The first clamping stage 800 is then in an unclamped state (not clamping the flexible elongate device 202). In this way, a net movement of the flexible elongate device 202 by a distance shown between X and Z can be achieved. As a result, the traction drive 324 has generated the retraction movement for the flexible elongate device 202.

The orifice 801 of the first clamping stage 800 also has an adjustable orientation about the longitudinal axis 820. The first clamping stage 800 is configured to cause the orifice 801 to rotate about the longitudinal axis 820, thereby adjusting the orientation of the orifice 801. For example, referring to FIG. 8A, when the flexible elongate device 202 is clamped by the first clamping stage 800, the first clamping stage 800 may cause the orifice 801 to rotate about the longitudinal axis 820 by a number of degrees. As a result, the flexible elongate device 202 is also rotated about the longitudinal axis by the same number of degrees.

FIG. 8B illustrates a traction drive 324 according to some embodiments. The traction drive 324 comprises the first clamping stage 800 and the first driving stage 810 of FIG. 8A. The traction drive 324 further comprises a second clamping stage 830 configured to releasably clamp the flexible elongate device 202. The second clamping stage 830 may also comprise an orifice 831, where the orifice 831 has an adjustable aperture and an adjustable orientation, as detailed above with respect to the first clamping stage 800. The traction drive 324 further comprises a second driving stage 840 which is configured to move the second clamping stage 830 along the longitudinal axis 820 of the flexible elongate device 202.

According to some embodiments of the present application, the clamping and releasing of the first clamping stage 800 and the second clamping stage 830 and the movement generated by the first driving stage 810 and the second driving stage 840 may be coordinated to move, or inchworm, the flexible elongate device 202 in a direction along the longitudinal axis 820.

For example, referring to FIG. 8B, to begin the first clamping stage 800 and the second clamping stage 830 may be in non-clamping states (not clamping the flexible elongate device 202). The first clamping stage 800 may clamp the flexible elongate device 202 by adjusting the aperture of the orifice 801. With the second clamping stage 830 still in a non-clamping state, the first driving stage 810 may move the first clamping stage 800 along a longitudinal axis 820 of the flexible elongate device 202 to move the first clamping stage 800 from a position X1 to a position Y1.

Following the movement from X1 to Y1, the second clamping stage 830 may clamp the flexible elongate device 202 by adjusting the aperture of the orifice 831. The first clamping stage 800 may release the flexible elongate device 202, so that the first clamping stage 800 is in a non-clamping state. With the first clamping stage 800 in a non-clamping state, the second driving stage 840 may then cause the second clamping stage 830 to move along the longitudinal axis 820 to move the second clamping stage 830 from a position X2 to a position Y1. In this way, a net movement of the flexible elongate device 202 by a distance which is the sum of the distance between X1 to Y1 and the distance between X2 to Y2 has been achieved. As a result, the traction drive 324 has generated the insertion movement for the flexible elongate device 202.

Subsequently, simultaneously or previously to the movement of the second clamping stage 830, the first clamping stage 800, while in a non-clamping state, may be repositioned, for example back to position X1. Subsequently, simultaneously or previously to the repositioning of the first clamping stage 800, for example back to X1, the second clamping stage 830, while in a non-clamping state, may be repositioned, for example back to position X2. Repositioning the first clamping stage 800 and the second clamping stage 830 enables repetition of the described steps for further insertion of the flexible elongate device 202 by movement of the first clamping stage 800 or the second clamping stage 830 along the longitudinal axis 820.

Table 1 below outlines movements of the clamping stages 800 830 via driving stages 810 840 that may be used to configure the traction drive 324 to move the flexible elongate device 202 along the longitudinal axis 820 in a direction towards the procedural site (distal). For each step in Table 1, the state in which the first clamping stage 800 and the second clamping stage 830 should be in for execution of the step are given.

| | | State | State of |
|---|---|---|---|
| | | of first | second |
| | | clamping | clamping |
| | Movement Step | stage 800 | stage 830 |
| 1 | Move first clamping stage 800 | Clamped | Non-clamped |
| | from X1 to Y1 | state | state |
| 2 | Move second clamping stage 830 | Non-clamped | Clamped |
| | from X2 to Y2 | state | state |
| 3 | Move first clamping stage 800 | Non-clamped | Clamped |
| | from Y1 to X1 | state | state |
| 4 | Move second clamping stage 830 | Clamped | Non-clamped |
| | from Y2 to X2 | state | state |
| 5 | Repeat 1-4 until desired movement | | |
| | has been achieved. | | |

While Table 1 illustrates example movement steps of the clamping stage 800 and 830, these steps are not intended to be limiting and it will be clear to the skilled person that modifications can be made to the steps in Table 1. For example, following step 3, step 1 may be repeated before implementing step 4 followed by step 2.

Furthermore, the steps in Table 1 may be modified to configure the traction drive 324 to move the flexible elongate device 202 along the longitudinal axis 820 in a direction away from the procedural site (proximal). Referring to FIG. 8B, when the second clamping stage 830 is in a non-clamped state, the first driving stage 810 may cause the first clamping stage 800 to move from a position X1 to a position Z1. The first clamping stage 800 may then release the flexible elongate device 202 so that the first clamping stage 800 is in a non-clamped state. The second clamping stage 830 may then clamp the flexible elongate device 202. With the first clamping stage 800 in a non-clamped state, the second driving stage 840 may cause the second clamping stage 830 to move from a position X2 to a position Z2. In this way, a net movement of the flexible elongate device 202 by a distance which is the sum of the distance between X1 to Z1 and the distance between X2 to Z2 has been achieved. As a result, the traction drive 324 has generated a retraction movement for the flexible elongate device 202.

As described for the insertion movement, the first clamping stage 800 and the second clamping stage 830 may then be repositioned to X1 and X2 respectively to allow for the movements to be repeated.

The orifices 801 831 of the first clamping stage 800 and the second clamping stage 830 respectively also have adjustable orientations about the longitudinal axis 820. The first clamping stage 800 is configured to rotate the orifice 801 about the longitudinal axis 820, thereby adjusting the orientation of the orifice 801. The second clamping stage 830 is configured to rotate the orifice 831 about the longitudinal axis 820, thereby adjusting the orientation of the orifice 801. In some embodiments, the first clamping stage 800 and the second clamping stage 830 are configured to rotate the orifices 801 831 simultaneously. For example, referring to FIG. 8B, when the flexible elongate device 202 is clamped by the first clamping stage 800 and the second clamping stage 830, the first clamping stage 800 and the second clamping stage 830 may cause the orifice 801 to rotate about the longitudinal axis 820 by a number of degrees. As a result, the flexible elongate device 202 is also rotated about the longitudinal axis 202 by the same number of degrees. In some embodiments, the first clamping stage 800 and the second clamping state 830 may alternatingly cause the flexible elongate device 202 to rotate. More specifically, the first clamping stage 800 may cause the flexible elongate device 202 to rotate while the second clamping stage 830 is in a non-clamped state, and the second clamping stage 830 may cause the flexible elongate device 202 to rotate when the first clamping stage is in a non-clamped state. The rotation of the flexible elongate device 202 may be performed simultaneously or non-simultaneously with the insertion/retraction of the flexible elongate device 202.

The traction drive 324 as described in relation to FIG. 8B, thus, enables unlimited insertion/retraction and/or roll of the flexible elongate device 202. As a result, the size of the traction drive 324 can be minimized, which further minimizes the space that the traction drive 324 would take in a procedure room.

In some embodiments, the total operating length of the traction drive 324 (the length along which the clamping stages 800 830 can move) is kept as short as possible to reduce the wasted tool channel length.

In some embodiments, the first driving stage 810 and the second driving stage 840 may comprise an actuator. In some embodiments, the first driving stage 810 and the second driving stage 840 may be linear drive mechanism, such as a linear bearing and an actuator with a ball screw. In some embodiments, the first driving stage 810 and the second driving stage 840 have enough force to provide for insertion of the flexible elongate device 202 into a patient.

In FIG. 8B, the detection zone 406 is defined over a section of the traction drive 324 in which both the flexible elongate device 202 passes through the orifices 801 831 of the first clamping stage 800 and the second clamping stage 830. Alternatively, the detection zone may be defined over a section of the traction drive 324 in which the flexible elongate device 202 passes through the orifice 801 of the first clamping stage 800 or the orifice 831 of the second clamping stage 830. In any of the above, when the flexible elongate device 202 is in a clamped state, using the first clamping stage 800 and/or the second clamping stage 830, a mechanical signature may be imprinted on the flexible elongate device 202 by the first clamping stage 800 and/or the second clamping stage 830 and a detection zone 406 may be defined. By knowing the location of the mechanical signature on the flexible elongate device 202 and the position of the first clamping stage and second clamping stage (for example from the steps in Table 1) a spatial relationship between the flexible elongate device 202 and the traction drive 324 is determined. By identifying a length of the flexible elongate device 202 within the detection zone 406, the control system 112 can determine a length of the flexible elongate device 202 distal to the traction drive 324 and/or a length of the flexible elongate device 202 proximal to the traction drive 324.

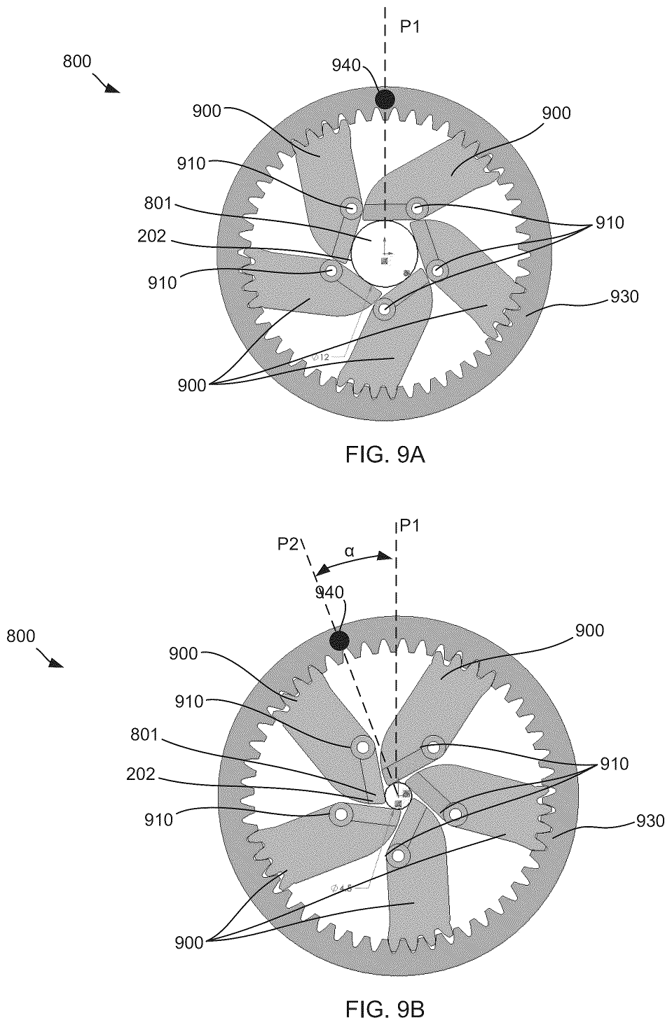

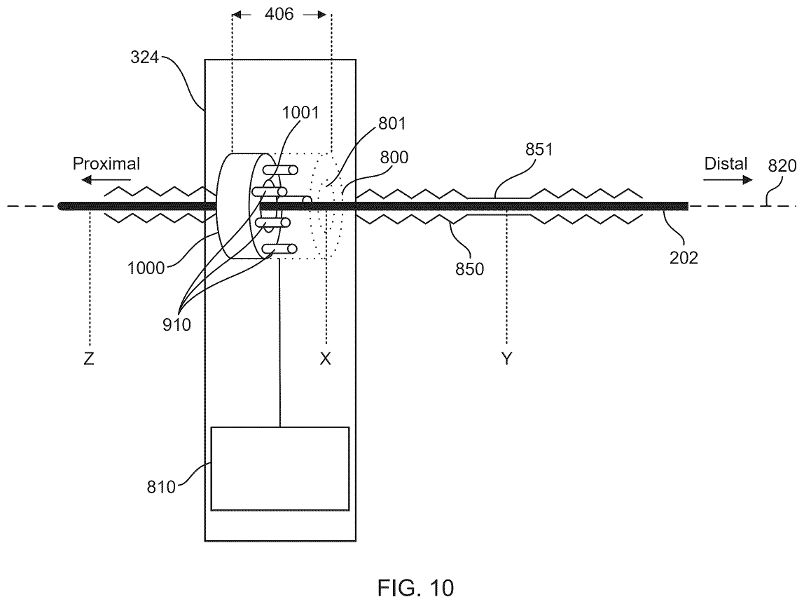

FIG. 9A and FIG. 9B illustrate a clamping stage 800 according to some embodiments, where the clamping stage 800 may be the first clamping stage 800 or the second clamping stage 830 described previously. The clamping stage 800 comprises a plurality of shutter blades 900 that are arranged to form the orifice 801. The circle illustrated within the orifice 801 indicates a cross-section of the flexible elongate device 202. While the flexible elongate device 202 is illustrated as having a circular cross-section, it will be clear to the skilled person that the flexible elongate device 202 may have alternative cross section shapes and dimensions. For example, the flexible elongate device 202 in FIG. 9A is illustrated as having a larger diameter than the flexible elongate device 202 in FIG. 9B.

Each shutter blade 900 is configured to couple to and rotate about a fixed pin 910. The clamping stage 800 further comprises an aperture adjustment member 930 configured to mechanically engage with the shutter blades 900. In some embodiments, aperture adjustment member 900 is shown as being a gear wheel with a number of teeth, and each shutter blade 900 is shown as having a number of teeth at one end configured to engage with the teeth of the gear wheel. However, other means of engagement between the aperture adjustment member 930 and the shutter blades 900 can be used.

The aperture adjustment member 930 is configured to drive the shutter blades 900 to control a size of the aperture of the orifice 801. In FIG. 9A, the aperture of the orifice 801 is shown as being a first size, for example to accommodate a flexible elongate device 202 of a first diameter. When the aperture adjustment member 930 is rotated anti-clockwise, each blade rotates about its associated fixed pin 910, causing the aperture of the orifice 801 to get smaller, as shown in FIG. 9B. In FIG. 9B, the aperture of the orifice 801 is shown as being a second size, smaller than the first size of FIG. 9A, for example to accommodate a flexible elongate device 202 of a second diameter. The change in the aperture of the orifice 801 between FIG. 9A and FIG. 9B is achieved by rotating the aperture adjust member 930 anticlockwise by a given amount.

To aid with discussion of the clamping stage 800 of FIG. 9A and FIG. 9B, a marker 940 has been illustrated on the aperture adjustment member 930. The marker 940 is only for illustrative purposes and has no limiting effect on the embodiments of FIG. 9A and FIG. 9B. In FIG. 9A, the marker 940 is shown as being in a first position P1 relative to the center of the orifice 801. In FIG. 9B, the aperture adjustment member 930 has been rotated anticlockwise so that the marker 940 has moved from the first position P1 relative to the center of the orifice to a second position P2 relative to the center of the orifice 801. The marker 940 has been rotated by an angle of a degrees from the first position P1 to the second position P2.

Conversely, starting from FIG. 9B, when the aperture adjustment member 930 is rotated clockwise, each blade 900 rotates about its associated fixed pin 910, causing the aperture of the orifice 801 to get larger, as shown in FIG. 9A.

For a given arrangement of blades 900 and aperture adjustment member 930, a movement of the aperture adjustment member 930 can be translated into a known change in diameter, or dimension, of the aperture of the orifice 801. In this way, a dimension, or diameter, of the aperture of the orifice 901 may be controlled.

While FIG. 9A and FIG. 9B show a fixed number of blades 900, and a given ratio of teeth between the aperture adjustment member 930 and each blade 900, other numbers of blades 900 and ratios of teeth can be used. Furthermore, while FIG. 9A and FIG. 9B illustrate a shape of the blades 900, it will be clear to the reader that other shaped blades 900 could be used.

In FIG. 9A and FIG. 9B, the longitudinal axis 820 is not shown, but is in the center of the circle indicating the flexible elongate device 202. The blades 900 are illustrated as being substantially in a single plane perpendicular to the longitudinal axis 820. In some embodiments, the blades 900 may be stacked in different planes, each plane perpendicular to the longitudinal axis 820. In some embodiments, the blades 900 may be arranged in an iris shutter arrangement.

In some embodiments, the clamping stage 800 further comprises an aperture adjustment actuator (not shown) to drive the aperture adjustment member 930. In the embodiments of FIG. 9A and FIG. 9B, the aperture adjustment member 930 is a ring and the aperture adjustment drive is a cable drive configured to rotate the aperture adjustment member 930 in an anti-clockwise or clockwise direction.

In some embodiments, the clamping stage 800 further comprises an aperture rotation member configured to mechanically engage with the shutter blades 900 and to drive the shutter blades 900 to control the adjustable orientation of the orifice 801, as subsequently discussed.

FIG. 10 illustrates the aperture rotation member 1000 of the clamping stage 800, according to some embodiments. In FIG. 10 parts of the clamping stage 800 are shown as transparent (with a dotted line) so as to allow the feature of the aperture rotation member 1000 to be clearly seen. In FIG. 10, the transparent part of clamping stage 800 may be considered to house the shutter blades 900 and the aperture adjustment member 930.

In an embodiment, the aperture rotation member 1000 is configured to mechanically couple to the clamping stage 800. Each pin 910 (also illustrated in FIGS. 9A and 9B) of the clamping stage 800 is fixed at a first end to the aperture rotation member 1000 and mechanically engages the shutter blades (not shown in FIG. 10) at a second end. The aperture rotation member 1000 does not block the passing of the flexible elongate device 202 through the traction drive 324. In FIG. 10, the aperture rotation member 1000 is illustrated as being a ring with an orifice 1001, so that the flexible elongate member 202 can pass through the orifice 1001. When the clamping stage 800 is in a clamped state and clamping the flexible elongate device 202, the aperture rotation member 1000 may be rotated to drive the shutter blades 900 via the pins 910. The driving of the shutter blades 900 in this manner causes the rotation of the aperture adjustment member 930. This may result in a rotation of the orifice 801 without a change in the aperture dimensions, if certain conditions are met. Specifically, the aperture rotation member 1000 and the aperture adjustment member 930 may be jointly rotated in a coordinated motion (the same rotation synchronously applied to both the aperture rotation member 1000 and the aperture adjustment member 930 throughout the joint rotation) to result in a rotation of the orifice 801 without a change in the aperture dimensions. In this way, the traction drive 324 can clamp onto a flexible elongate device 202 and rotate it.

To enable clamping or release of the flexible elongate device 202, the aperture adjustment member 930 is rotated when the aperture rotation member 1000 stays static (does not rotate). This causes the shutter blades 900 to rotate about the pins 910 to enable the aperture of the orifice 801 to be adjusted and cause the clamping stage 800 to clamp the flexible elongate device.

While the clamping stage is in a clamped state and clamping the flexible elongate device 202, both the aperture adjustment member 930 and the aperture rotation member 1000 are rotated simultaneously. As the pins 910 are moving at a same rate as the shutter blades 900, the shutter blades 900 do not rotate about the pins 910, and so the aperture of the orifice 801 is not changed. However, the orientation of the aperture of the orifice 801 is changed as it is rotated about the longitudinal axis 202.

In FIG. 10, the aperture rotation member 1000 is illustrated as being centered on the longitudinal axis 820 and positioned more proximal to the procedural site than the shutter blades 900 (not shown) and aperture adjustment member 930 (not shown) as indicated by the transparent portion of the clamping stage 800. However, in some embodiments the aperture rotation member 1000 may be placed on an axis parallel to the longitudinal axis 820. In some embodiments, the aperture rotation member 1000 may be placed more distal to the procedural site than the shutter blades 900 (not shown) and aperture adjustment member 930 (not shown) as indicated by the transparent portion of the clamping stage 800.

In some embodiments, the clamping stage 800 further comprises an aperture orientation actuator configured to drive the aperture rotation member 1000. In the embodiments of FIG. 10, the aperture rotation member 1000 is a ring and the aperture orientation actuator is a cable drive configured to rotate the aperture rotation member 1000 in an anti-clockwise or clockwise direction.

In FIG. 10, the aperture rotation member 1000 is illustrated as being a ring with an orifice 1001 that aligns with orifice 801 along the longitudinal axis 202, so that the flexible elongate member 202 can pass through the clamping stage 800. However, other configurations of the aperture rotation member 1000 can be used, so long as the pins 910 are fixed by the aperture rotation member 1000 while allowing the flexible elongate device 202 to pass through the clamping stage 800.

As illustrated in FIG. 8A, FIG. 8B and FIG. 10, in some embodiments, the traction drive 324 optionally includes a tubular bellow section 850 that encloses the flexible elongate device 202 where it passes through the traction drive 324. The tubular bellow section 850 establishes a barrier that prevents contamination of the traction drive 324 by the flexible elongate device 202 and vice versa. In some embodiments, the tubular bellow section 850 comprises one or more grip regions 851 that are designed to facilitate engagement of the clamping stage(s) with the tubular bellow section, e.g., without crushing the tubular bellow section. The grip region may be an interruption of the folded bellow design that enables lengthening and shortening of the tubular bellow section. The interruption may be a smooth section (without the folding) that enables gripping without crushing of elements of the tubular bellow section. One grip region may be provided for each clamping stage. No grip regions are visible in FIGS. 8A and 8B because the grip regions are occluded by the clamping stages. One grip region is visible in FIG. 10, in a region of the bellow section 850, where the second clamping stage would be placed. Different diameter bellow sections may be used to accommodate different sized flexible elongate devices (for example, a large gastroscope versus a lung biopsy catheter). An element (e.g., an RFID tag) may be used to identify its style (diameter) to the traction drive 324 or to a system comprising the traction drive 324.

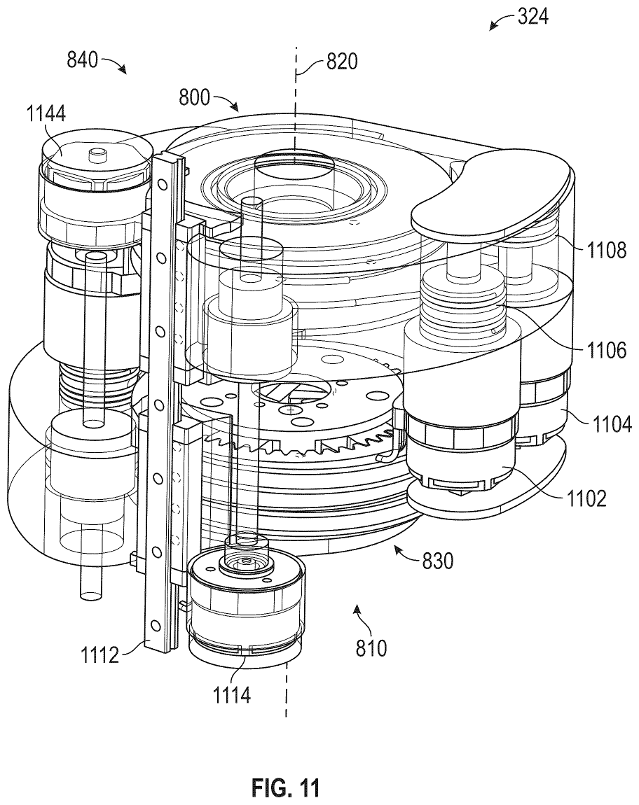

FIG. 11 shows an implementation of a traction drive according to some embodiments. Similar to the previously described traction drives, the traction drive 324 includes a first clamping stage 800, a second clamping stage 830, a first driving stage 810, and a second driving stage 840.

The clamping stages 800, 830 both include an aperture adjustment actuator 1102 and an aperture orientation actuator 1104 (shown for the first clamping stage 800 but not for the second clamping stage 830). The aperture adjustment actuator 1102 drives the aperture adjustment member 930, e.g., as illustrated in FIGS. 9A and 9B. In some embodiments, the aperture adjustment actuator 1102 drives the aperture adjustment member 930 via cable 1106. The aperture orientation actuator 1104 drives the aperture orientation member 1000, e.g., as illustrated in FIG. 10. In some embodiments, the aperture orientation actuator 1104 drives the aperture orientation member 1000 via cable 1108. While a cable-based implementation is shown, the disclosure is not limited to this configuration. Other transmission elements such as belts, gears, etc. may be used.

The operation of the aperture adjustment actuator 1102 and the operation of the aperture orientation actuator 1104 may be coordinated to configure the clamping stages 800, 830 in different manners. Specifically, by rotating the aperture adjustment member and/or the aperture orientation member in coordination, the behaviors as described in reference to FIGS. 9A, 9B, and 10 are achieved. As previously discussed, these behaviors include maintaining a clamped or non-clamped state while performing/not performing a rotation. These behaviors further include a transition from the clamped to the non-clamped state, or from the non-clamped state to the clamped state while no rotation is performed, or during an ongoing rotation. As discussed, all of these behaviors may be accomplished by controlling absolute rotations of the aperture adjustment member and the aperture rotation member, and a relative rotation of the aperture adjustment member and the aperture rotation member. For example, rotation of the aperture adjustment member and the aperture orientation member in the same direction rotates the entire clamp without changing aperture size and can be used after gripping the flexible elongate device to rotate the flexible elongate device. Rotation of the aperture adjustment member and the aperture orientation member in opposite directions (or one with respect to the other nonmoving) changes aperture size and can be used to release or grip catheter.

The first and second driving stages 810, 840 together comprise a linear rail 1112. The first and the second clamping stages 810, 840 both ride on the linear rail 1112, enabling translational movement along longitudinal axis 820. The translational movement of the first and the second clamping stages 810, 840 is controlled by a first and a second driving stage actuator 1114, 1144. The first and second driving stage actuators 1114, 1144 may be ball screw actuators or other types of linear actuators.

The described traction drive 324 may be included in the embodiments described above, such as in relation to FIG. 3 and/or FIG. 4.

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM), a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

What is claimed is:

1. A medical system comprising:
a manipulator assembly comprising:
a traction drive for driving a flexible elongate device of a medical instrument along an insertion axis of the manipulator assembly, wherein the traction drive is configured to form a detection zone that imprints a mechanical signature on the flexible elongate device;
a shape sensor configured to measure a shape of the flexible elongate device; and
a control system coupled to the manipulator assembly, the control system configured to:
identify, using the shape sensor, the mechanical signature at a location of the flexible elongate device, and
determine, based on the location, a spatial relationship between the flexible elongate device and the traction drive.

2. The medical system of claim 1, wherein the flexible elongate device is in a slack configuration proximal to the traction drive.

3. The medical system of claim 2, wherein the manipulator assembly further comprises an instrument carriage, and wherein a proximal end of the flexible elongate device terminates at the instrument carriage.

4. The medical system of claim 3, wherein the instrument carriage is configured to move in a direction towards the traction drive.

5. The medical system of claim 1, wherein the control system is further configured to:
determine a position of the flexible elongate device in an anatomical reference frame using the spatial relationship between the flexible elongate device and the traction drive, based on a registration of the flexible elongate device to an anatomical model.

6. The medical system of claim 5, wherein the control system is further configured to: determine an insertion depth of the flexible elongate device using the spatial relationship between the flexible elongate device and the traction drive, and using the registration.

7. The medical system of claim 1, wherein the traction drive comprises a plurality of rollers forming a friction drive for the driving of the flexible elongate device.

8. The medical system of claim 7, wherein the plurality of rollers is spatially arranged to imprint the mechanical signature.

9. The medical system of claim 8, wherein the plurality of rollers is arranged in a pattern so as to form a straight section of the flexible elongate device.

10. The medical system of claim 8, wherein the plurality of rollers is arranged in a pattern so as to form a zig-zagging section of the flexible elongate device.

11. The medical system of claim 7, wherein a surface of the plurality of rollers is textured to imprint the mechanical signature.

12. The medical system of claim 1, wherein the traction drive comprises:
a first clamping stage configured to releasably clamp the flexible elongate device, the first clamping stage comprising:
an orifice for the flexible elongate device, wherein the orifice has an adjustable aperture and an adjustable orientation; and
a first driving stage configured to move the first clamping stage along a longitudinal axis of the flexible elongate device.

13. A non-transitory machine-readable medium comprising a plurality of machine-readable instructions executed by one or more processors associated with a medical system, the plurality of machine-readable instructions causing the one or more processors to perform a method comprising:
identifying, using a shape sensor configured to measure a shape of a flexible elongate device, a mechanical signature at a location of the flexible elongate device, the mechanical signature being imprinted on the flexible elongate device by a detection zone of a traction drive comprised in a manipulator assembly, wherein the traction drive is configured to driving the flexible elongate device of a medical instrument along an insertion axis of the manipulator assembly; and
based on the location, determining a spatial relationship between the flexible elongate device and the traction drive.

14. The non-transitory machine-readable medium of claim 13, wherein the method further comprises:
moving an instrument carriage at which a proximal end of the flexible elongate device terminates towards the traction drive.

15. The non-transitory machine-readable medium of claim 13, wherein the method further comprises:
determining a position of the flexible elongate device in an anatomical reference frame using the spatial relationship between the flexible elongate device and the traction drive, based on a registration of the flexible elongate device to an anatomical model.

16. The non-transitory machine-readable medium of claim 15, wherein the method further comprises: determining an insertion depth of the flexible elongate device using the using the spatial relationship between the flexible elongate device and the traction drive, and using the registration.

17. A method for operating a medical system, the method comprising:
identifying, using a shape sensor configured to measure a shape of a flexible elongate device, a mechanical signature at a location of the flexible elongate device, the mechanical signature being imprinted on the flexible elongate device by a detection zone of a traction drive comprised in a manipulator assembly, wherein the traction drive is configured to driving the flexible elongate device of a medical instrument along an insertion axis of the manipulator assembly; and
based on the location, determining a spatial relationship between the flexible elongate device and the traction drive.

18. The method of claim 17, further comprising:
moving an instrument carriage at which a proximal end of the flexible elongate device terminates towards the traction drive.

19. The method of claim 17, further comprising:
determining a position of the flexible elongate device in an anatomical reference frame using the spatial relationship between the flexible elongate device and the traction drive, based on a registration of the flexible elongate device to an anatomical model.

20. The method of claim 19, further comprising: determining an insertion depth of the flexible elongate device using the using the spatial relationship between the flexible elongate device and the traction drive, and using the registration.
